(19) United States
(12) Patent Application Publication
(10) Pub. No.: US 2023/0270484 A1
(43) Pub. Date: Aug. 31, 2023

(54) SYSTEM AND METHOD FOR ENHANCED-REALITY ELECTROSURGICAL SYSTEM

(71) Applicant: Jerome Canady Research Institute for Advanced Biological and Technological Sciences, Takoma Park, MD (US)

(72) Inventors: Jerome Canady, Lakeland, FL (US); Changhan Jun, Beltsville, MD (US); Taisen Zhuang, Rockville, MD (US)

(21) Appl. No.: 18/195,164

(22) Filed: May 9, 2023

Related U.S. Application Data
(63) Continuation-in-part of application No. 17/471,828, filed on Sep. 10, 2021.
(60) Provisional application No. 63/339,631, filed on May 9, 2022.
(60) Provisional application No. 63/076,771, filed on Sep. 10, 2020.

(51) Int. Cl. A61B 18/04

(52) U.S. Cl. CPC A61B 18/042; A61B 2018/00636

(57) ABSTRACT

An electrosurgical system and method for control of an electrosurgical system with an enhanced reality display system wirelessly connected to the gas-enhanced electrosurgical system. The electrosurgical system comprises a gas-enhanced electrosurgical generator, a voice-recognition system connected to said gas-enhanced electrosurgical generator, a robotic system, and an enhanced reality display system wirelessly connected to said gas-enhanced electrosurgical system. The gas-enhanced electrosurgical generator may comprise a power module, a gas control module, and a control module, wherein said control module is configured to provide voice control of said power module and said gas module.

### CROSS-REFERENCE TO RELATED APPLICATIONS

The present application is a continuation-in-part of U.S. Non-provisional patent application Ser. No. 17/471,828 filed on Sep. 20, 2021, which claims the benefit of the filing date of U.S. Provisional Patent Application Ser. No. 63/076,771 filed on Sep. 20, 2020. The present application further claims the benefit of the filing date of U.S. Provisional Patent Application Ser. No. 63/339,631 filed by the present inventors on May 9, 2022.

The aforementioned patent applications are hereby incorporated by reference in their entirety.

### BACKGROUND OF THE INVENTION

### Field of the Invention

The present invention relates to gas-enhanced electrosurgical systems, and more particularly, to a system and method for voice-control of a gas-enhanced electrosurgical system and other electronic operating room equipment combined with an augmented reality display system.

### Brief Description of the Related Art

A variety of different electrosurgical generators are known. U.S. Pat. No. 4,429,694 to McGreevy disclosed an electrosurgical generator and argon plasma system and a variety of different electrosurgical effects that can be achieved depending primarily on the characteristics of the electrical energy delivered from the electrosurgical generator. The electrosurgical effects included pure cutting effect, a combined cutting and hemostasis effect, a fulguration effect and a desiccation effect. Fulguration and desiccation sometimes are referred to collectively as coagulation.

Another method of monopolar electrosurgery via argon plasma technology was described by Morrison in U.S. Pat. No. 4,040,426 in 1977 and McGreevy U.S. Pat. No. 4,781,175. This method, referred to as argon plasma coagulation (APC) or argon beam coagulation is a non-contact monopolar thermoablative method of electrocoagulation that has been widely used in surgery for the last twenty years. In general, APC involves supplying an ionizable gas such as argon past the active electrode to target tissue and conducting electrical energy to the target tissue in ionized pathways as non-arcing diffuse current. Canady described in U.S. Pat. No. 5,207,675 the development of APC via a flexible catheter that allowed the use of APC in endoscopy. These new methods allowed the endoscopist to combine standard monopolar electrocautery with a plasma gas for coagulation of tissue.

Yet another system is disclosed in U.S. Patent Application Publication No. 2013/0296846, which disclosed a system for simultaneously cutting and coagulating tissue. Another system, referred to as a “cold atmospheric plasma” system, is disclosed in U.S. Patent Application Publication No. 2014/0378892.

Several different systems and methods for performing Cold Atmospheric Plasma (CAP) treatment have been disclosed. For example, U.S. Pat. No. 10,213,614 discloses a two-electrode system for CAP treatement of cancer cells.

Another exemplary Cold Atmospheric Plasma system is disclosed in U.S. Pat. No. 9,999,462. The disclosed system has two units, namely a Conversion Unit (CU) and a Cold Plasma Probe (CPP). The Conversion Unit is connected to high frequency electrosurgical generator (ESU) output and converts the ESU signal to a signal appropriate for performing cold atmospheric plasma procedures. The Cold Plasma Probe is connected to the Conversion Unit output. At the end of the Cold Plasma Probe cold plasma is produced and is thermally harmless to living tissue, i.e., it cannot cause burns to the tissue. This cold plasma, however, is deadly for cancer cells while leaving normal cells unaffected. The disclosed Cold Plasma Conversion Unit is unique in that it utilizes a high voltage transformer to up-convert the voltage (1.5-50 kV), down-convert the frequency (<300 kHz), and down-convert the power (<30 W) of the high-voltage output from an electrosurgical unit (U.S. Pat. No. 9,999,462).

Further, various systems and methods for controlling gas flow and an integrated gas-assisted electrosurgical generator having a graphical user interface is disclosed in WO2018/191265, entitled “Electrosurgical Gas Control Module” and WO2019199281, entitled “Gas Enhanced Electrosurgical Generator.”

Various additional systems and methods for assisting surgeons in controlling surgical systems have been disclosed, including the following:
U.S. Published Patent Application No. 20210037176, entitled “Control Device, Medical Observation System, Control Method, And Computer Readable Recording Medium”;
U.S. Published Patent Application No. 20210035575 entitled “Electronic Apparatus, And Method Of Controlling To Execute Function According To Voice Command Thereof”;
U.S. Published Patent Application No. 20210012775 entitled “Electronic Device And Voice Recognition Control Method Of Electronic Device”;
U.S. Published Patent Application No. 20190365488 entitled “Surgical Assistance System And Method For Generating Control Signals For Voice Control Of A Surgery Assistance System Robot Kinematics That Can Be Moved In A Motor-Controlled Manner”;
U.S. Published Patent Application No. 20190038236 entitled “Medical Voice Command Integration”;
U.S. Published Patent Application No. 20160338570 entitled “Medical System”; U.S. Published Patent Application No. 20100131280 entitled “Voice Recognition System For Medical Devices”;
U.S. Published Patent Application No. 20210011294 entitled “Augmented Visualization During Surgery”;
U.S. Published Patent Application No. 20200372714 entitled “Augmented Reality Medical Diagnostic Projection”;
U.S. Published Patent Application No. US20200352655 entitled “Methods, Devices, And Systems For Augmented Reality Guidance Of Medical Devices Into Soft Tissue”;

U.S. Published Patent Application No. 20200219324 entitled “Surgeon Head- Mounted Display Apparatuses”;

U.S. Published Patent Application No. 20200211420 entitled “System And Method For Three-Dimensional Augmented Reality Guidance For Use Of Medical Equipment”; and

U.S. Published Patent Application No. 20200107904 entitled “System And Method For Virtual Reality Data Integration And Visualization For 3d Imaging And Instrument Position Data”.

### SUMMARY OF THE INVENTION

In a preferred embodiment, the present invention is an electrosurgical system and method for control of an electrosurgical system with an enhanced reality display system wirelessly connected to the gas-enhanced electrosurgical system. The electrosurgical system comprises a gas-enhanced electrosurgical generator, a voice- recognition system connected to said gas-enhanced electrosurgical generator, a robotic system, and an enhanced reality display system wirelessly connected to said gas-enhanced electrosurgical system. The gas-enhanced electrosurgical generator may comprise a power module, a gas control module, and a control module, wherein said control module is configured to provide voice control of said power module and said gas module.

In another embodiment, the present invention is a method for operating an electrosurgical system using an enhanced reality display.

Still other aspects, features, and advantages of the present invention are readily apparent from the following detailed description, simply by illustrating preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow chart illustrating a method for voice activation of electronic equipment in an operating room in accordance with a preferred embodiment of the present invention.

FIG. 2 is a detailed flow diagram illustrating a method for voice activation of electronic equipment in an operating room in accordance with a preferred embodiment of the present invention.

FIG. 3 is a diagram of a system for voice activation of electronic equipment in an operating room in accordance with a preferred embodiment of the present invention.

FIG. 4A is a block diagram of a cold atmospheric plasma generator of a preferred embodiment of the present invention.

FIG. 4B is a block diagram of a plasma generator of an alternate preferred embodiment of the present invention.

FIG. 4C is a block diagram of a plasma generator of another alternate preferred embodiment of the present invention.

FIG. 4D is a block diagram of an integrated gas-enhanced electrosurgical generator having a plurality of gas modules of another alternate preferred embodiment of the present invention.

FIG. 5 is a perspective view of an integrated gas-enhanced electrosurgical generator of a preferred embodiment of the present invention.

FIG. 6 is a block diagram illustrating an electrosurgical system having an enhanced- reality display in accordance with a preferred embodiment of the present invention.

FIG. 7 is a flow chart illustrating a pre-op method in accordance with a preferred embodiment of the present invention.

FIG. 8 is a flow chart illustrating an operation method in accordance with a preferred embodiment of the present invention.

### DETAILED DESCRIPTION OF THE PREFERRED EMBODIMENTS

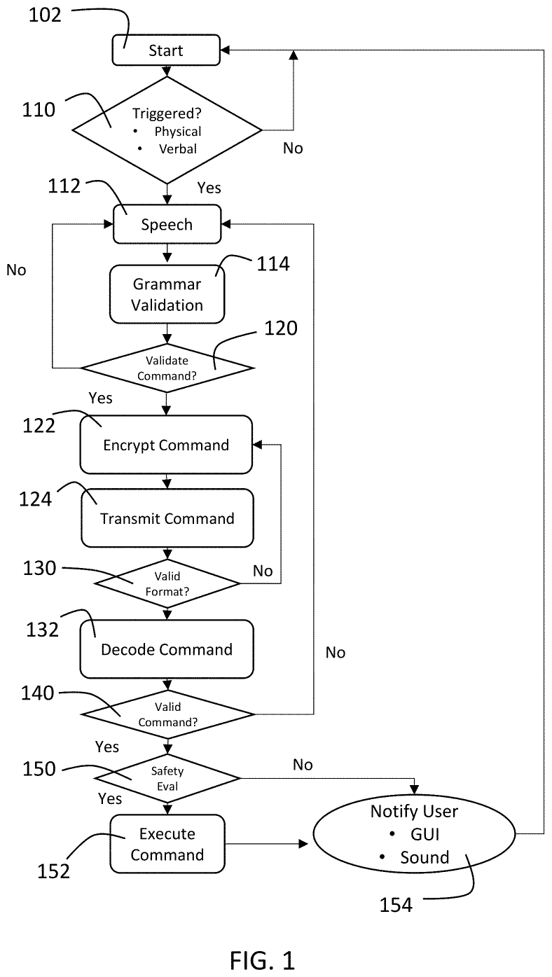

A method for voice activation of electronic equipment in an operating room in accordance with a preferred embodiment of the present invention is described with reference to FIG. 1. The method starts 102 with the voice-control system being activated or turned on. Once active or on the voice control system can be triggered 110 through physical or verbal cues or prompts. If a trigger event 110 is detected, the voice control system uses speech recognition software 112 to identify voice instructions. The grammar of the detected speech is then validated 114. If the speech is not validated as a command the system returns to the speech recognition step 112 and/or causes the system to notify the user visually or audibly that the command was not validated or returns to the detection of a new trigger 110. If a command is validated 120, the command is encrypted 122 and transmitted 124 to the electronic operating room equipment to which the command is directed.

The transmitted encrypted commend is received at the electronic equipment, which decodes the comments 132 and determines whether the decoded command is valid 130. If the decoded command is valid, the electronic equipment performs a safety evaluation 150 to ensure that the command can be safely executed. If the decoded command is deemed to be safe, the command is executed by the electronic equipment and the user is notified verbally or visually that the command has been executed. If the command is not deemed to be safe, the user is notified 154 visually or verbally.

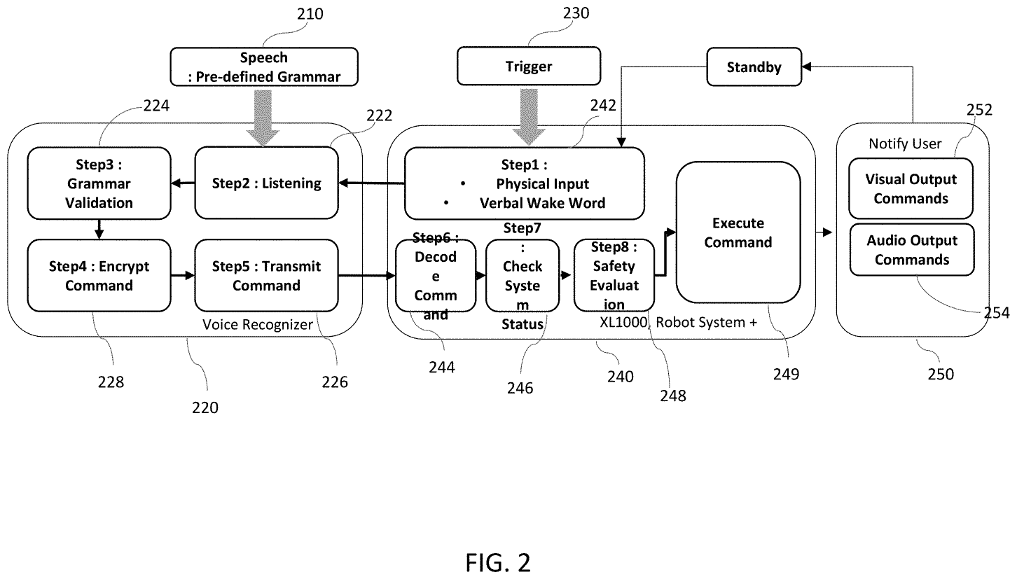

A method for voice activation of electronic equipment in an operating room in accordance with a preferred embodiment of the present invention is further shown in the detailed flow diagram of FIG. 2.

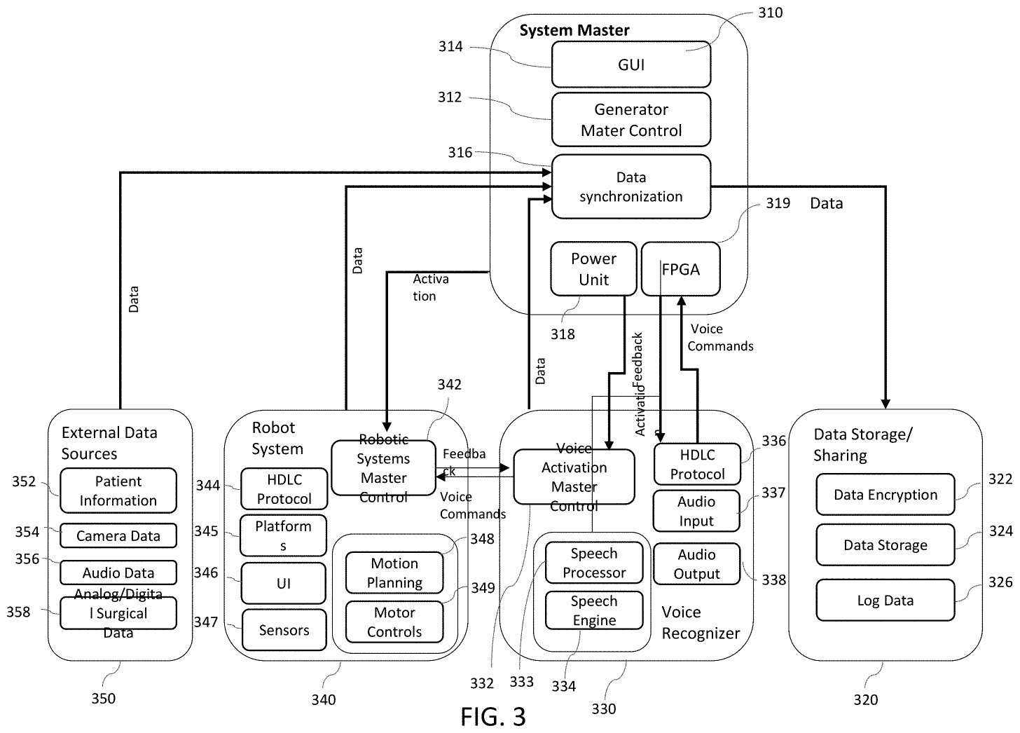

Further, a system for voice activation of electronic equipment in an operating room in accordance with a preferred embodiment of the present invention is shown in FIG. 3.

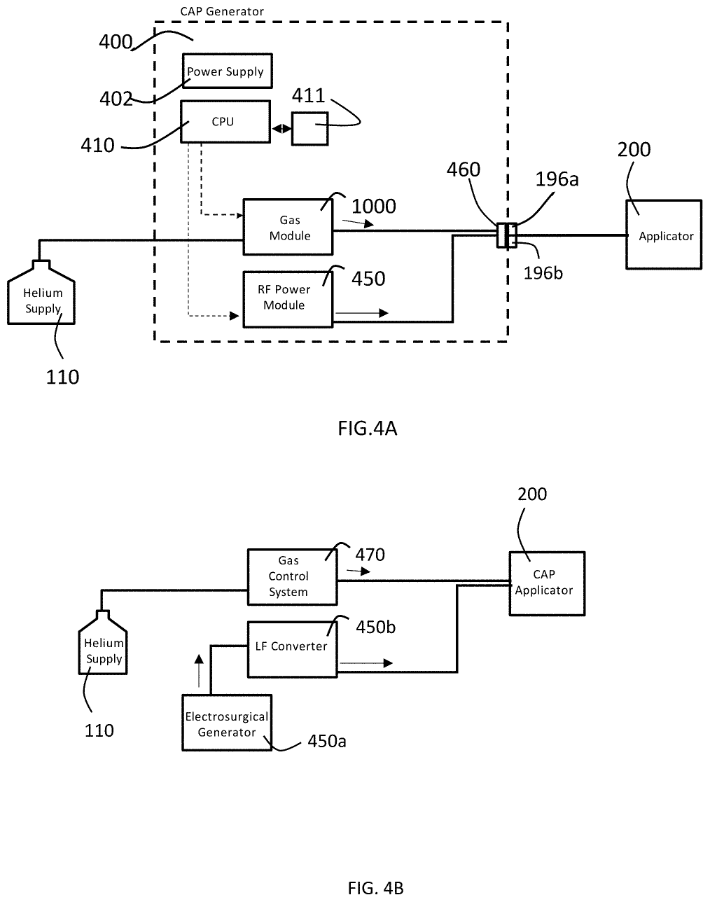

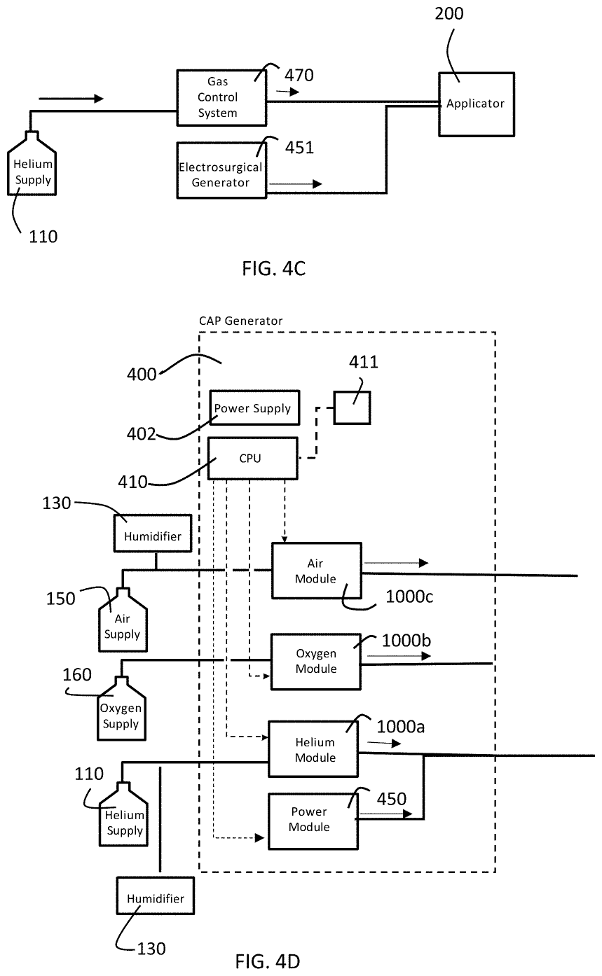

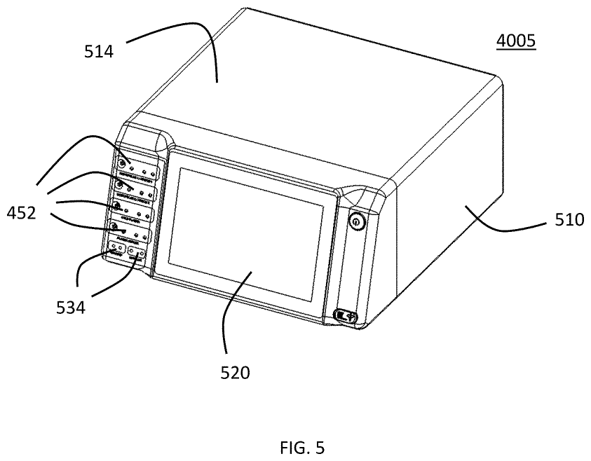

The system and method of the present invention may be used with a variety of electronic equipment used in an operating room. One such system is a cold atmospheric plasma system. As shown in FIG. 4A, an exemplary cold atmospheric plasma (CAP) generator 400 has a power supply 402, a CPU (or processor or FPGA) 410 and a memory or storage 411. The system further has a display 520 (FIG. 5), which may be the display of a tablet computer. The CPU 410 controls the system and receives input from a user through a graphical user interface displayed on display 520. The CAP generator further has a gas control module 1000 connected to a source 410 of a CAP carrier gas such as helium. The CAP generator 400 further has a radio frequency (RF) power module 450 for generating radio frequency (RF) energy. The RF power module contains conventional electronics such as are known for providing RF power in electrosurgical generators. The RF Power module operates with a frequency between 10-200 kHz and output peak voltage from 3 kV to 6 kV and preferably at a frequency near (within 20%) of 40 Hz, 100 Hz or 200 Hz. The gas module 1000 and RF power module 450 are connected to connector 460 that allows for CAP joint mixer 200 (or a CAP applicator 1100 in FIGS. 11A and 11B) to be connected to the generator 400 via a connector having an electrical connector 196a and gas connector 196b.

As shown in FIG. 4B, other arrangements for delivery of the carrier gas and the electrical energy may be used with the invention. In FIG. 4B, a source 110 of a carrier gas (helium in this example) is provided to a gas control system 470 of any type, which supply the gas at a controlled flow rate to CAP joint mixer 200. A conventional electrosurgical generator 450a supplies high frequency (HF) energy to a low frequency converter 450b, which outputs electrical energy having a frequency in the range of 10 kHz to 200 kHz and an output voltage in the range of 3 kV to 6 Kv.

Another embodiment, shown in FIG. 4C, has a carrier gas source 110 connected to a conventional gas control system 470, which in turn is connected to the CAP joint mixer 200, and a conventional electrosurgical generator 451 also connected to the CAP joint mixer 200.

A housing 500 for a CAP-enabled gas-enhanced electrosurgical generator 500 in accordance with a preferred embodiment of the present invention is shown in FIG. 5. The gas-enhanced generator 500 has a housing 510 made of a sturdy material such as plastic or metal similar to materials used for housings of conventional electrosurgical generators. The housing 510 has a removable cover 514. The housing 510 and cover 514 have means, such as screws, tongue and groove, or other structure for removably securing the cover to the housing. The cover 514 may comprise just the top of the housing or multiple sides, such as the top, right side, and left side, of the housing 510. The housing 510 may have a plurality of feet or legs attached to the bottom of the housing. The bottom of the housing 510 may have a plurality of vents for venting from the interior of the gas-enhanced generator.

On the face of the housing 514 there is a touch-screen display 520 and a plurality of connectors 532, 534 for connecting various accessories to the generator, such as an argon plasma probe, a hybrid plasma probe, a cold atmospheric plasma probe, or any other electrosurgical attachment. The face of the housing 510 is at an angle other than 90 degrees with respect to the top and bottom of the housing 510 to provide for easier viewing and use of the touch screen display 520 by a user. One or more of the gas control modules may be mounted within a gas-enhanced electrosurgical generator 500.

The CAP-enabled gas-assisted electrosurgical generator has a graphical user interface (GUI) for controlling the components of the system using the touch screen display 520. The graphical user interface for example, may control robotics, argon-monopolar cut/coag, hybrid plasma cut, cold atmospheric plasma, bipolar, plasma sealer, hemo dynamics or voice activation. The graphical user interface further may be used with fluorescence-guided surgery. The graphical user interface (GUI) further may be used with guided imaging such as CT, MRI, or ultrasound. The graphical user interface may communicate with RFID (such as may be found in various electrosurgical attachments) and may collect and store usage data in a storage medium. The graphical user interface communicates with the field-programmable gate array (“FPGA”), which may control an irrigation pump, insufflator, full bridge for adjusting the power output, fly back for regulating the power (DC to AC) and a foot pedal. The GUI further communicates with a database of data with associated predicted CAP settings or dosages via the CPU 410. The database storage may be internal memory or other internal storage 411 or external storage.

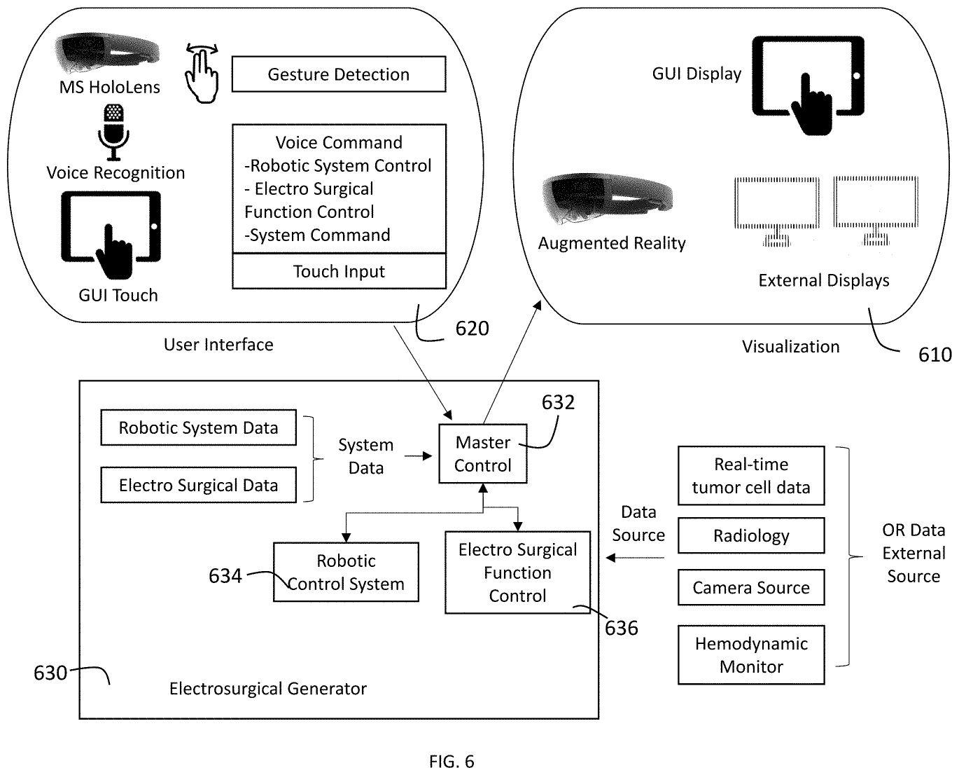

FIG. 6 is a block diagram illustrating an electrosurgical system having an enhanced- reality display in accordance with a preferred embodiment of the present invention. The system may have various components 610 for visualization of the surgical procedure, such as graphical user interface (GUI), external displays and an augmented reality display. The system further may have various input means, such as voice recognition, GUI touchscreen, and gesture detection. Still further, the system has an electrosurgical generator 630, which has a master control 632 that may be a processor or group of processors. The master control 632 communicates with a robotic control system 634 and an electrosurgical function control 636 and receives system data from those systems and uses that data to control the system. The master control 632 also may receive data, such as real-time tumor cell data, radiology, camera(s), and a hemodynamic monitor data, from external sources.

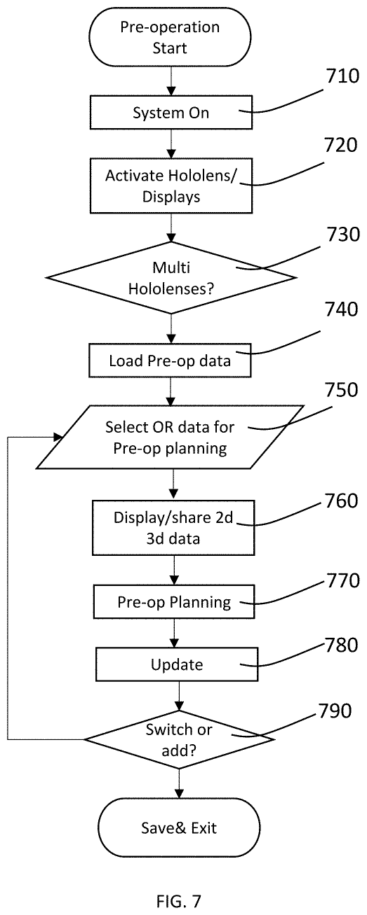

FIG. 7 is a flow chart illustrating a pre-op method in accordance with a preferred embodiment of the present invention. The system is turned on 710, one or more hololenses (augmented reality display) is activated, 720, the system detects the one or more hololenses 730, pre-op data 740 is loaded into the master controller, operating room (OR) data is selected from pre-op planning 750, the hololens(es) or other displays share 2d and/or 3 data 760, pre-op planning is performed 770, and system is updated with the pre-op planning 780, the system adds the pre-op planning to storage or returns 790 to the selection of different OR data for pre-op planning.

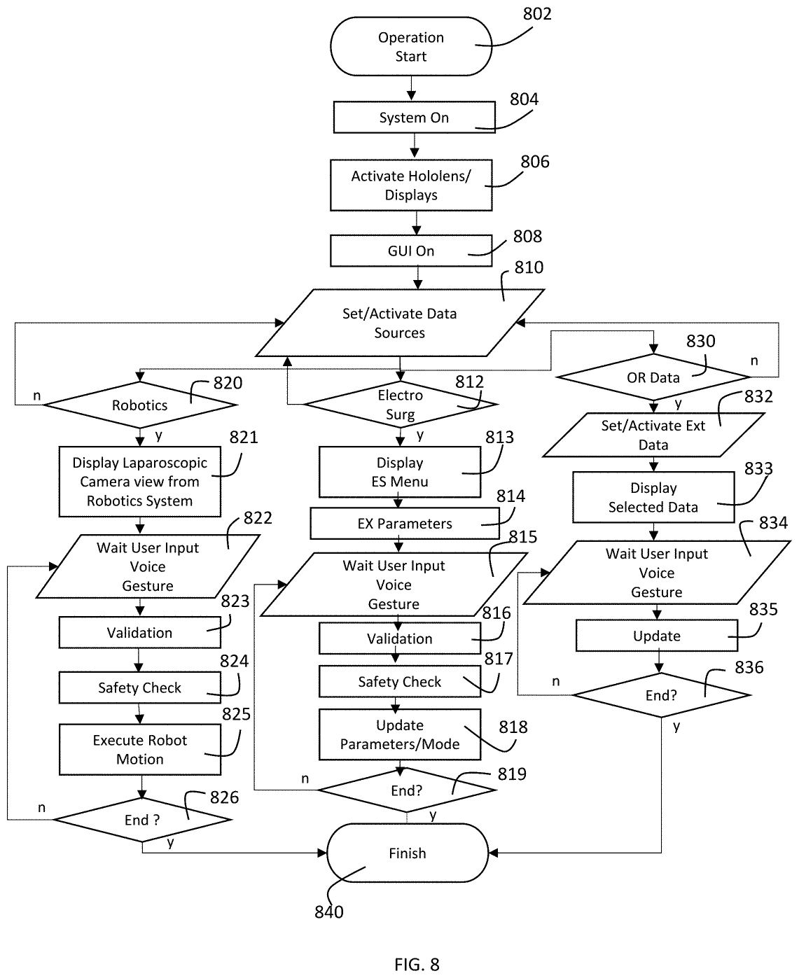

FIG. 8 is a flow chart illustrating an operation method in accordance with a preferred embodiment of the present invention. The procedure states 802 with the system being turned on 804. The hololens/displays are activated 804. The graphical user interface is turned on 808. The system or user sets/activated various data sources 810, such as surgical robotics 820, electrosurgical system 812. And OR data 830. With respect to a robotics system, for example, a laparoscopic camera view may be displayed on the hololens 821, the robotics system may then wait for user input such as a voice commend 822, the system then validates the voice command 823, performs a safety check 824, and if that is passed, executes a robot motion associated with the voice command 825. With respect to the electrosurgical system (ES), the ES menu is displayed on the hololens or other display 813, parameters are selected 814, and user voice comments are awaited. 815. When a voice command for the ES is received, the system performs validation 816, a safety check 817, and if the safety check is passed, updates the parameters or mode in accordance with the voice instruction. 818. With respect to OR data 830, external data is set or activated 832, the selected data is displayed on the hololens 833, user voice input is awaited 834, and the OR data is updated when use input is received 835. The various processes end (826, 189, 836) once the data is updated.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. An electrosurgical system comprising:
gas-enhanced electrosurgical generator;
a voice-recognition system connected to said gas-enhanced electrosurgical generator;
a robotic system system; and
an enhanced reality display system wirelessly connected to said gas-enhanced electrosurgical system.

2. An electrosurgical system according to claim 1, wherein said gas-enhanced electrosurgical generator comprises:
a power module;
a gas control module; and
a control module, wherein said control module is configured to provide voice control of said power module and said gas module.

3. A method for operating an electrosurgical system using an enhanced reality display.
